(19) United States
(12) Patent Application Publication
(10) Pub. No.: US 2012/0064027 A1
(43) Pub. Date: Mar. 15, 2012

(54) Absorbable adhesive paste

(76) Inventor: Shalaby W. Shalaby, Anderson, SC (US)

(21) Appl. No.: 12/807,575

(22) Filed: Sep. 9, 2010

(51) Int. Cl. A61K 31/765; A61P 7/04; A61P 31/04; A61P 17/02

(52) U.S. Cl. 424/78.37

(57) ABSTRACT

An absorbable adhesive paste contains absorbable solid carboxy-bearing microparticles dispersed in a continuous phase of an alkoxyalkyl cyanoacrylate. The paste may contain a soluble absorbable polymeric viscosity modifier and at least one bioactive agent to maximize the effective application as a cover for compromised tissues and wounds or as sealants, blocking agents, or hemostatic agents.

The present application claims the benefit of prior U.S. Ser. No. 61/276,130, filed Sep. 9, 2009.

### FIELD OF THE INVENTION

The present invention is directed to an absorbable adhesive paste containing absorbable microparticles of at least one carboxyl-bearing polyester, dispersed in a cyanoacrylate-based continuous phase of an alkoxyalkyl cyanoacrylate. The flow properties of the paste can be modulated using a soluble absorbable polymeric viscosity modifier to allow its application as moderately viscous paste or extruded ribbon to function as a hemostatic agent in bleeding wounds or a cover for burns and ulcers, with polymerization occurring upon contacting the respective wet tissue.

### BACKGROUND OF THE INVENTION

Prior to the mid-1990s, the non-absorbable, hydrolytically stable n-butyl and isobutyl cyanoacrylates were acknowledged as the most effective type of tissue adhesives in terms of adhesive joint strength. They have been used as hemostatic sealants as well as blocking agents for treating vascular aneurysms. In spite of the availability of other tissue adhesives, such as gelatin-resorcinol, those cyanoacrylates retained their competitive edge because of their fast polymerization and strength of their adhesive joints. The dominance of those cyanoacrylates was recently threatened by growing interest in biodegradable biomacromolecule-based systems, such as (1) fibrin glue; (2) mussel adhesive protein; (3) prolamine gel, a biodegradable protein; and (4) transforming growth factor beta (TGF-β). However, none of those adhesives could provide the adhesive joint strength required in many applications, and hence remained mostly as tissue sealants or carriers of bioactive drugs. This and the growing interest in synthetic absorbable polymers prompted Shalaby and co-workers to explore the development of the absorbable cyanoacrylate-based systems (U.S. Pat. Nos. 5,350,798; 6,299,631; 6,723,114; 7,083,634; 7,348,426). The latter prior art dealt primarily with liquid absorbable tissue adhesives containing methoxypropyl cyanoacrylate, stabilizers to prevent premature polymerization, and absorbable polymeric modifiers to modulate the liquid viscosity and increase the compliance of the cured tissue adhesives. Meanwhile, additional prior art on the cyanoacrylate tissue adhesives dealt with the use of (1) Lewis acids as stabilizers against premature anionic polymerization (U.S. Ser. No. 11/479,424); (2) stabilizers against premature free radical polymerization (U.S. Ser. No. 11/240,277); (3) a hardening component consisting of a low molecular weight derivative prepared by modifying one carboxyl group in a di- or tricarboxylic acid of the citric acid cycle with an electron-withdrawing group (U.S. Ser. No. 10/543,156); (4) an adhesive composition containing a mixture of two monomer species having different absorption rates (U.S. Ser. No. 09/919,877; and (5) a biological or therapeutic agent (U.S. Pat. No. 7,238,828). Additionally, on two occasions, the prior art dealt with the cyanoacrylate monomers as part of two-phase systems that are different from the traditional liquid tissue adhesive. More specifically, the prior art dealt with (1) a dual-phase polymeric agent-delivery composition comprising a continuous biodegradable hydrogel phase, a discontinuous particulate phase comprising defined microparticles, and a bioactive agent to be delivered contained in at least said discontinuous particulate phase (U.S. Pat. No. 6,589,549), and (2) a cyanoacrylate adhesive composition in thixotropic paste form that is made by mixing liquid cyanoacrylate ester with powdered non-absorbable organic fillers selected from polycarbonates, polyvinylidene fluorides, polyethylenes and acrylic block copolymer resins containing saturated elastomer segments (U.S. Pat. No. 4,105,715).

Reviewing the prior art discussed above shows that with the exception of the teaching of U.S. Pat. No. 4,105,715 on cyanoacrylate adhesive paste all other disclosures were directed to liquid cyanoacrylate systems with or without soluble polymeric viscosity modifiers or polymerized cyanoacrylate in a hydrogel matrix. Moreover, U.S. Pat. No. 4,105,715 discloses a novel means of converting liquid cyanoacrylate to a non-drip thixotropic gel or paste through incorporating certain finely divided non-absorbable organic powders that are not dissolved or fully dissolved in the cyanoacrylate monomer. The long-standing interest in absorbable cyanoacrylate tissue adhesives, the use of low molecular weight acidic stabilizers against premature anionic polymerization, and the recent growing interest in adhesive paste, prompted the pursuit of the study, subject of the instant invention. Accordingly, the present invention is directed to a liquid precursor of an absorbable polycyanoacrylate mixed with microparticles of an absorbable polyester, which not only converts the liquid precursor into a paste, but also acts as a stabilizer against premature anionic polymerization and its acidic degradation, with by-products displaying antimicrobial activity.

### SUMMARY OF THE INVENTION

In general, the present invention is directed to an absorbable adhesive paste of a composition of from about 20 to about 70 percent by weight of absorbable microparticular carboxyl-bearing absorbable polyester dispersed in a continuous phase of at least one cyanoacrylate monomer, the continuous phase including at least 50 percent by weight of an alkoxyalkyl cyanoacrylate, wherein the microparticular carboxyl-bearing absorbable polyester is an acid-terminated polyglycolide.

A major aspect of the instant invention deals with an absorbable adhesive paste comprising a composition of from about 20 to about 70 percent by weight of absorbable microparticular carboxyl-bearing absorbable polyester dispersed in a continuous phase of at least one cyanoacrylate monomer, the continuous phase comprising at least 50 percent by weight of an alkoxyalkyl cyanoacrylate, wherein the microparticular carboxyl-bearing absorbable polyester comprises acid-terminated polyglycolide, and wherein the alkoxyalkyl cyanoacrylate comprises methoxypropyl cyanoacrylate, and wherein the absorbable polyester comprises from about 40 to about 60 percent by weight and the cyanoacrylate comprises from about 60to about 40 percent by weight of the composition, and further, wherein the continuous phase comprises from about 60 to about 98 percent by volume of methoxypropyl cyanoacrylate and from about 40 to about 2 percent by volume of ethyl cyanoacrylate.

A specific aspect of the invention deals with an absorbable adhesive paste comprising a composition of from about 20 to about 70 percent by weight of absorbable microparticular carboxyl-bearing absorbable polyester dispersed in a continuous phase of at least one cyanoacrylate monomer, the continuous phase comprising at least 50 percent by weight of an alkoxyalkyl cyanoacrylate, wherein the said composition further comprises at least one absorbable polymeric additive to modulate the paste viscosity and adhesive joint strength of the cured paste, and wherein the at least one polymeric additive is selected from the group consisting of acylated polyethylene glycols, absorbable amorphous polyether-ester urethanes and their acylated derivatives, absorbable crystalline polyether-ester-urethanes and their acylated derivatives, crystalline polyether-esters and their acylated derivatives, and absorbable segmented polyaxial copolyesters and their acylated derivatives.

A clinically important aspect of the instant invention deals with an absorbable adhesive paste comprising a composition of from about 20 to about 70 percent by weight of absorbable microparticular carboxyl-bearing absorbable polyester dispersed in a continuous phase of at least one cyanoacrylate monomer, the continuous phase comprising at least 50 percent by weight of an alkoxyalkyl cyanoacrylate, wherein said composition is in the form of an antibacterial hemostatic agent and cover for burns, ulcers, and contaminated wounds, and can further comprise at least one bioactive agent selected from the group consisting of antimicrobial agents, anti-inflammatory agents, thrombogenic agents, anesthetic agents, antineoplastic agents, and tissue growth-promoting agents.

### DETAILED DESCRIPTION OF PREFERRED EMBODIMENTS

Since the early development of non-absorbable cyanoacrylate tissue adhesives, which were dominantly represented by butyl cyanoacrylates, they were described as adhesives, sealants, and blocking agents, with or without bioactive agents. However, maximizing their effectiveness in the latter three clinical applications was limited by the physicochemical properties of the liquid monomers that are difficult-to-apply precisely onto the application sites, which has been partially addressed by using soluble absorbable polymeric viscosity modifiers in certain absorbable tissue adhesives with minimum effect on the adhesive joints strength thereof Accordingly, the instant invention focuses on the use of essentially insoluble absorbable microparticles as a dispersed phase in a monomeric liquid continuous phase to modulate its rheological properties without compromising the stability of the monomer(s) against premature polymerization. Further, the liquid monomers of the prior art are exceptionally susceptible to premature anionic polymerization in the presence of trace amounts of water and weak basic agents, which limits their shelf-stability and interferes with their deployment, without curing, into the application sites as in the case of their use as blocking agents of diseased biological conduits in vascular and urinogenital systems. This has been addressed to a limited extent by using acid or acid generating additives, with poorly predictable performance, to retard or prevent premature anionic polymerization. Accordingly, the instant invention teaches the novel use of polymeric absorbable microparticles with carboxyl-bearing constituent chains as solid acid stabilizers against premature anionic polymerization. Still further, the liquid monomers of the prior art are not well-suited to use as sealants or covers on compromised tissues, particularly those presenting large surface areas, due to the excessive heat generation associated with the monomer(s) conversion to polymers, which in turn, can lead to tissue necrosis. Accordingly, the instant invention deals with the use of reduced amounts of the monomer by being a component of a paste containing 20-70 percent of solid, non-heat-generating absorbable microparticles and hence, reducing the amount of heat generation at the application sites as in burns and ulcers and large area wounds. Still further, the prior art liquid monomers are not well-suited for use as absorbable covers for burns and ulcers for limited residence tie and/or can be removed at will whenever the need arises, without mechanical damage to the biological sites because of their high adhesive joint strength. Accordingly, the instant invention teaches the use of a paste, which forms an adhesive joint with precisely modulated strength, based on the nature of the application site, by controlling the weight fraction of the monomer in the paste, i.e., less monomer leads to weaker adhesive joint and less damage to the application site. Still further, the liquid monomers of the prior art are not suited for incorporating bioactive agents, such as antimicrobial, anti-inflammatory or anesthetic agents, peptides, proteins, tissue growth promoters, that are hydrated or carry hydroxylic, amine, and/or primary amide groups, which essentially can cause an immediate, premature conversion of the monomers to polymers. Accordingly, the instant invention provides for the use of carboxyl-bearing microparticles with carboxyl-terminated polymeric chains upon which several types of the aforementioned bioactive agents can be immobilized on their surfaces and below the surface to allow the controlled release of the bioactive agents without compromising the intended properties of the adhesive paste. Finally, the prior art liquid monomers are not suited for use effectively to achieve a timely hemostasis in large area wounds, in sites with flowing blood or body fluids, and traumatic wounds in battle fields or accidents because the texture of the liquid adhesive, which makes it difficult to apply and retain position to assist the granulation step during wound healing. Accordingly, the instant invention is directed to the use of a paste which minimizes or eliminates the undesirable features of the liquid free-flowing tissue adhesive, while inherently exhibiting antimicrobial activity to reduce the likelihood of infection at the application site.

In concert with the above noted discussion, the instant invention deals with novel adhesive paste compositions associated with unprecedented performance as mild adhesives, highly effective protective covers or sealants for wounds and ulcers as well as easy-to-administer blocking agents.

Further illustrations of the present invention are provided by the following examples:

### EXAMPLE 1

### Preparation of Absorbable Carboxyl-Bearing Microparticles: A Typical Method

The preparation of a carboxyl-terminated absorbable polyester, such as carboxyl-terminated polyglycolide (C-PG), typically entailed the polymerization of glycolide in the presence of glycolic acid as the initiator and stannous octanoate as the catalyst as described in U.S. Pat. Nos. 5,714,159 and 6,413,539, both of which are hereby incorporated herein by reference. The ratio of monomer-to-initiator was varied to control the molecular weight of the polymer and hence, concentration of the carboxylic group per unit weight, which, in turn, is reflected in maintaining the melting temperature of the polymer between 210° C. and 222° C. The polymer was ground to particles having a diameter of about 0.1 mm and then purified as described in the referenced U.S. patents. Jet milling was then used to reduce the particle size of the ground powder to achieve microparticles having an average diameter between 2 and 12 microns.

### EXAMPLE 2

### Preparation and Evaluation of Typical Adhesive Pastes Using 2-Methoxypropyl Cyanoacrylate, AP-M

Typical pastes were made by mixing mechanically predetermined amounts of a C-PG from Example 1 with 2-methoxypropyl cyanoacrylate (MPC) in a laminar flow hood. Different paste compositions were prepared, the adhesive joint strengths of the cured pastes were evaluated using the fabric peel test described earlier [Flagle, J. et al., Trans. Soc. Biomater, 22 376 (1999)]. The composites of the adhesive pastes and their average (using 4 test specimens per set) adhesive joint strength data measured shortly after preparation and upon aging at 50° C. for 1 week are summarized in Table I.

TABLE I
Composition and Properties of Typical AP-M-type Adhesive Pastes

| Adhesive Paste | Weight Ratio of | Average Adhesive Joint Strength (N) | |
|---|---|---|---|
| Number | C-PG/MPC | Initial | After Aging @ 50° C. |
| AP-M1 | 60/40 | 1.36 | 1.71 |
| AP-M2 | 55/45 | 1.55 | 1.95 |
| AP-M3 | 50.50 | 4.09 | 4.2 |

Although the present invention has been described in connection with the preferred embodiments, it is to be understood that modifications and variations may be utilized without departing from the principles and scope of the invention, as those skilled in the art will readily understand. Accordingly, such modifications may be practiced within the scope of the following claims. Moreover, Applicant hereby discloses all subranges of all ranges disclosed herein. These subranges are also useful in carrying out the present invention.

What is claimed is:

1. An absorbable adhesive paste comprising a composition of from about 20 to about 70 percent by weight of absorbable microparticular carboxyl-bearing absorbable polyester dispersed in a continuous phase of at least one cyanoacrylate monomer, the continuous phase comprising at least 50 percent by weight of an alkoxyalkyl cyanoacrylate.

2. An absorbable adhesive paste as in claim 1 wherein the microparticular carboxyl-bearing absorbable polyester comprises acid-terminated polyglycolide.

3. An absorbable adhesive paste as in claim 1 wherein the microparticular carboxyl-bearing absorbable polyester comprises acid-terminated polyglycolide, wherein the alkyoxyalkyl cyanoacrylate comprises methoxypropyl cyanoacrylate, and wherein the absorbable polyester comprises from about 40 to about 60 percent by weight and the cyanoacrylate comprises from about 60to about 40 percent by weight of the composition.

4. An absorbable adhesive paste as in claim 1 wherein the continuous phase comprises from about 60 to about 98 percent by volume of methoxypropyl cyanoacrylate and from about 40 to about 2 percent by volume of ethyl cyanoacrylate.

5. An absorbable adhesive paste as in claim 1 further comprising at least one absorbable polymeric additive to modulate the paste viscosity and adhesive joint strength of the cured paste.

6. An absorbable adhesive paste as in claim 5 wherein the at least one polymeric additive is selected from the group consisting of acylated polyethylene glycols, absorbable amorphous polyether-ester urethanes and their acylated derivatives, absorbable crystalline polyether-ester-urethanes and their acylated derivatives, crystalline polyether-esters and their acylated derivatives, and absorbable segmented polyaxial copolyesters and their acylated derivatives.

7. An absorbable adhesive paste as in claim 1 in the form of an antibacterial hemostatic agent and cover for burns, ulcers, and contaminated wounds.

8. An absorbable adhesive paste as in claim 1 comprising at least one bioactive agent selected from the group consisting of antimicrobial agents, anti-inflammatory agents, thrombogenic agents, anesthetic agents, antineoplastic agents, and tissue growth-promoting agents.
